(19) United States
(12) Patent Application Publication
(10) Pub. No.: US 2022/0266054 A1
(43) Pub. Date: Aug. 25, 2022

(54) METHODS FOR IMPROVING SKIN APPEARANCE

(71) Applicant: ANGEL AESTHETICS, Denver, CO (US)

(72) Inventor: Angel MARTINEZ, Denver, CO (US)

(73) Assignee: ANGEL AESTHETICS, Denver, CO (US)

(21) Appl. No.: 17/632,156

(22) PCT Filed: Aug. 18, 2020
(86) PCT No.: PCT/US2020/046798

§ 371 Date: Feb. 1, 2022

Related U.S. Application Data
(60) Provisional application No. 62/889,724, filed on Aug. 21, 2019.

(51) Int. Cl. A61N 5/06; A61N 5/067; A61B 17/32; A61B 17/34

(52) U.S. Cl. CPC A61N 5/0616; A61N 5/067; A61B 17/32; A61B 17/34; A61N 2005/0651; A61B 2017/320004; A61B 2017/00893

(57) ABSTRACT

The disclosure herein relates to methods for improving the appearance of skin, in particular facial skin. The method disclosed herein are designed to reduce the appearance of various skin conditions such as melasma, fine lines, and ruddiness due to natural aging and photoaging. More specifically, the present disclosure relates to a thee step process comprising light therapy, chemical peel, and dermaplane for improving the appearance of facial skin.

### RELATED APPLICATION

The present application claims priority to U. S. Provisional Patent Application Ser. No. 62/889,724, of same title, filed Aug. 21, 2019, the entire disclosure of which is incorporated herein by reference in its entirety for all purposes.

### TECHNICAL FIELD

The disclosure herein relates to methods for improving the appearance of skin, in particular facial skin. The method disclosed herein are designed to reduce the appearance of various skin conditions such as melasma, fine lines, and ruddiness due to natural aging and photoaging. More specifically, the present disclosure relates to treatment methods that target both the surface of the skin and below the surface of the skin. In one embodiment, the present disclosure relates to a thee step process comprising light therapy, chemical peel, and dermaplane for improving the appearance of facial skin.

### BACKGROUND

The skin is the largest organ of the body. It plays an important role in protecting the body by keeping out pathogens, preventing excessive water loss, providing insulation, regulating temperature, communicating environmental information, and synthesizing vitamins to name a few. The skin is composed of three primary layers, the epidermis, the dermis and the hypodermis. The epidermis is the outermost layer of the skin. It forms a waterproof, protective wrap over the body's surface. The main types of cells that make up the epidermis are keratinocytes, melanocytes, Langerhans cells, and Merkel cells. The dermis is the layer of skin beneath the epidermis. It consists of connective tissue and protects the body from stress and strain. The dermis also contains the hair follicles, sweat glands, sebaceous glands, apocrine glands, lymphatic vessels, blood vessels, and nerve endings. The hypodermis is beneath the dermis. The hypodermis consists of loose connective tissue, adipose tissue, and elastin that attaches the skin to the underlying bone and muscle. The main type of cells in the hypodermis are fibroblasts, macrophages, and adipocytes.

Many factors affect the health and appearance of the skin. Non-limiting factors include, age, genetics, environmental factors, allergies/sensitivities, nutrition/diet, smoking, and lifestyle. As we age, skin can become rough, dry, and itchy, which may be due to the loss of sweat and oil glands. The loss of elastic and adipose tissue can cause fine lines, wrinkles, and laxity (i.e., sagging). Cartilage loss in the nose can cause the nasal tip to droop. Thinning of the skin can make the skin more transparent, fragile, and prone to developing skin lesions. On the environmental side, exposure to ultraviolet (UV) radiation from the sun is one of the primary factors of skin aging and is referred to as photoaging. The effects of photoaging include melasma, freckles, age spots, uneven skin color, loss of skin tone, increased number wrinkles, increased depth of wrinkles, actinic keratosis, texture changes, redness or blotchiness from broken blood vessels, skin cancer, and the like. With the rise in the aging population, there is a demand for anti-aging products that reduce the appearance of aging and photoaging.

### SUMMARY

The following simplified summary provides a basic understanding of some aspects of the claimed subject matter. This summary is not an extensive overview, and is not intended to identify key/critical elements or to delineate the scope of the claimed subject matter. Its purpose is to present some concepts in a simplified form as a prelude to the more detailed description that is presented below.

The disclosure herein is directed to a method for improving the appearance of skin. In particular, facial skin. The first step of the method is to evaluate the skin for various skin conditions as well as for other factors that can affect treatment such as scar tissue, moles, or tattoos. Exemplary skin conditions include, but are not limited to, acne, actinic keratosis, atopic dermatitis, broken capillaries, cherry anginomas, folliculitis barbae, pseudofolliculitis barbae, hyperpigmentation, keratosis pilaris, age spots, melisma, nevus, psoriasis, rosacea, scars, seborrheic dermatitis, skin tags, spider veins, wrinkles, and the like. The skin is also scored to determine the Fitzpatrick type. A skin treatment plan is developed to address as many of the skin conditions identified, while minimizing downtime. Exemplary skin treatments include, but are not limited to chemical peels, dermaplane, intense pulse light therapy, laser (both ablative and non-ablative) therapy, light emitting diode therapy, microdermabrasion, microneedling, oxygenation therapy, and the like. In general, the treatment plan should include a treatment that treats the surface as well as below the surface of the skin. For example, microdermabrasion, ablative lasers, chemical peels, microneedling, or oxygenation treat the surface of the skin whereas intense pulse light therapy, and non-ablative lasers treat below the surface of the skin. In general, the treatments that treat below the surface of the skin should be done before the treatment that treat the surface of the skin. In some cases, the subject will benefit from the use of a post treatment exfoliating therapy such as microdermabrasion or dermaplane. The post-treatment exfoliation therapy should occur between 3 days to 6-7 weeks after the skin treatments.

To the accomplishment of the foregoing and related ends, certain illustrative aspects are described herein in connection with the following description and the annexed drawings. These aspects are indicative, however, of but a few of the various ways in which the principles of the claimed subject matter may be employed and the claimed subject matter is intended to include all such aspects and their equivalents. Other advantages and novel features may become apparent from the following detailed description when considered in conjunction with the drawings

### BRIEF DESCRIPTION OF THE DRAWINGS

The present disclosure, in accordance with one or more various embodiments, is descried in detail with reference to the following figures. The drawings are provided for purposes of illustration only and merely depict typical or example embodiments of the disclosure. These drawings are provided to facilitate the reader's understanding of the disclosure and shall not be considered limiting of the breadth, scope, or applicability of the disclosure. It should be noted that for clarity and ease of illustration these drawings are not necessarily made to scale.

FIG. 1 is a before and after pictures of a subject undergoing the skin improvement treatments disclosed herein.

FIG. 2 is a before and after pictures of a subject undergoing the skin improvement treatments disclosed herein.

FIG. 3 is a before and after pictures of a subject undergoing the skin improvement treatments disclosed herein.

FIG. 4 is a before and after pictures of a subject undergoing the skin improvement treatments disclosed herein.

FIG. 5 is a before and after pictures of a subject undergoing the skin improvement treatments disclosed herein.

### DETAILED DESCRIPTION

This disclosure is directed to a method for improving the appearance of skin due to aging, photoaging, and/or skin condition. The method improves the appearance of skin by reducing or eliminating melisma, age spots, and/or freckles; improving skin texture; improving skin tone; reducing or eliminating fine lines and/or wrinkles; reducing or eliminating acne; and/or reducing pore size.

From time-to-time, the disclosure described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments

### Definitions

Unless defined otherwise, all technical terms used herein have the same meaning as is commonly understood by one of ordinary skill in the art to which this disclosure belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

In the description that follows, a number of terms are extensively utilized. The following non-limiting definitions provide a clear and consistent understanding of the specification and claims, including the exemplary scope to be given such terms.

When the terms “one,” “a,” or “an” are used in this disclosure, they mean “at least one” or “one or more,” unless otherwise indicated.

The terms “invention” or “present invention” as used herein are intended to be non-limiting and are not intended to refer to any single embodiment of the particular invention but encompasses all possible embodiments as described in the specification and the claims.

The term “treat” or “treatment” as used herein refers to a procedure provided to a subject to improve the appearance of at least one skin condition.

The term “at home” as used herein refers treatments/procedures that can be done at home, often using products available over-the-counter.

The term “in-office” as used herein refers to treatments/procedures that are done by a professional skin care specialist such as an esthetician or physician.

### Skin Conditions

The term “acne” as used herein refers to blocked oil glands and/or hair follicles. Acne includes pimples, blackheads, whiteheads, cysts, and nodules. Acne can occur anywhere on the skin with the face, back, and buttocks being notable problematic areas.

The term “actinic keratosis” as used herein refers to thick, warty, rough, reddish growths.

The term “aging skin” as used herein refers to the changes in the appearance of skin due to aging and environmental factors such as (but not limited to) sagging, fine lines and wrinkles, discoloration, dryness, broken capillaries, thinning, age spots, and the like.

The term “atopic dermatitis” or “eczema” as used herein refers to inflamed, red, dry, and itchy patches of skin. The patches can occur anywhere on the body, but most commonly are seen on the face, elbows, hands, and in skin folds.

The term “broken capillaries” or “broken blood vessels” or “telangiectasias” as used herein refers to dilated blood vessels.

The term “blue nevus” as used herein refers to blue, gray-blue, or brown-blue papules.

The term “cherry anginomas” as used herein refers to small, bright red domes created by dilated blood vessels.

The term “dry skin” or “xeroderma” as used herein refer to a condition when the skin does not retain sufficient moisture resulting in skin that is itchy, rough, scaling, or cracking.

The terms “fine lines,” “lines,” or “wrinkles” as used herein refers to elastic changes in tissue resulting in a crease or fold.

The term “folliculitis barbae” as used herein refers to an infection of the hair follicles often caused by bacteria such as Staphylococcus aureus.

The term “pseudofolliculitis barbae” or “razor bumps” as used herein refers to hair that curls back and grows into the skin.

The term “hyperpigmentation” or “uneven skin tone” as used herein refers to patches of skin that is darker in color than the normal surrounding skin.

The term “keratosis pilaris” as used herein refers to small, rough bumps on the skin that are typically red or white and generally do not hurt or itch.

The term “lentigines” “age spots” or “liver spots” as used herein refers to dark spots on the skin. Age spots are generally bigger than freckles.

The term “melasma” or “chloasma” or “pregnancy mask” as used herein refers to tan or brown patches on the skin, often on the cheeks, nose, forehead, and chin.

The term “nevus” or “nevi” or “moles” as used herein refers to brown or black spots that can be smooth, wrinkled, flat, raised, or may have hair growing from them.

The term “oily skin” as used herein refers to the overproduction of sebum from the sebaceous glands.

The term “psoriasis” as used herein refers to thick, red patches or plaques of skin covered with white or silvery scales. Psoriasis is caused by a person's immune system triggering the accelerated growth of new skin cells resulting in the accumulation of dead skin cells that form the patches or plaques.

The term “rosacea” as used herein refers to chronic swelling, flushing, and/or redness in the face, most commonly on the nose, chin, cheeks, and forehead. The skin may be thickened and/or have prominent or broken blood vessels.

The term “scar” as used herein refers to an area of fibrous tissue that replaces normal skin after an injury. There are different types of scars. Hypertrophic scars result from overproduced collagen which causes the scar to be raised above the surrounding skin. Keloid scars are scar tissue which continues to grow forming a frim, rubbery lesions or shiny, fibrous nodules. Atrophic scars form a sunken recess in the skin, which has a pitted appearance. Stretch marks or striae are scars that occur when the skin is stretched rapidly.

The term “sebaceous hyperplasia” as used herein refers to small bumps on the skin that are often skin colored, white, or slightly yellow and range in size from one to several millimeters.

The term “seborrheic dermatitis” or “dandruff” or “cradle cap” as used herein refers to rough scaly skin or the scalp and face.

The term “seborrheic keratosis” as used herein refers to black or brown raised spots, or wart-like growths.

The term “skin tags” as used herein refers to small growths of skin that have a raised surface. They are usually flesh-colored or slightly darker and hang from the skin by a stalk.

The term “spider veins” as used herein refers to damaged veins that form thin lines, webs, or branches on the surface of the skin.

The term “sun damage” or “photoaging” as used herein refers to the changes in skin due to exposure to ultraviolet light such as (but not limited to) sunburn, wrinkles, age spots, dry skin, actinic keratosis and the like.

The term “TMJ” or “temporomandibular joint disorder” as used herein refers to (but not limited to) pain or tenderness in the jaw, jaw joint, muscles that control jaw movement; pain in an around the ear, aching facial pain, or difficulty opening or closing the mouth.

### Skin Treatments

There are numerous skin treatments available for improving the appearance of skin. Some are available for at home use, while others require the treatment to be provided by a licensed practitioner or licensed physician (herein referred to as “in-office” treatments). In general, at home treatments tend to address one skin problem, such as fine lines/wrinkles, acne, or dry skin, and do not provide dramatic results that can be achieved with in-office treatments. In-office treatments include, but are not limited to, laser treatments for laser rejuvenation and laser resurfacing, microdermabrasion, Botox 0 (anabotulinumtoxin A), dermal fillers, chemical peels, light therapy (LED and/or pulse), microneedling, extractions, dermaplane, skin oxygenation, and hydrodermabrasion, to name a few.

A Chemical peel or chemexfoliation is a procedure in which a chemical solution is applied to the skin to rapidly exfoliate the skin. Chemical peels vary in the depth of exfoliation. Superficial peels use mild acids and only penetrate the outermost layer of skin. Medium peels penetrate the meddle and outer layer of skin and deep peels fully penetrate the middle layer of skin. Chemical peels are used to correct skin irregularities in texture, such as fine lines, and color, such as spots caused by sun damage. Common acids used in chemical-peel solutions include, but are not limited to: alphahydroxy acid (AHA), glycolic acid, lactic acid, salicylic acid, trichloroacetic acid (TCA), and phenol to name a few. Chemical peels can be self-neutralizing.

Dermaplane is a simple exfoliating treatment that helps restore smoothness in the skin, removes dead skin cells and promotes product absorption.

Extractions are performed to help clear clogged pores. These are best on a regular basis and especially when experiencing breakouts. The extraction process involves a steam or mask to loosen pores and then removal.

Hair removal uses lasers to eliminate or reduce the growth of hair in a specific area. The type of laser used depend on the skin tone and the texture of the hair. Alexandrite lasers are generally used for those with light skin. Ruby lasers are generally used for those with light or find hair and fair skin. Diode lasers are generally used for fair to medium skin tones. Nd and LP Nd: YAG laser is generally use for darker skin tones.

Intense pulse light (IPL) therapy uses intense pulses of visible light in the range of 400 to 1200 nanometers. IPL uses filters to narrow the light wavelength to target specific structures and chromohores (e.g. melanin, red blood cells, etc.). IPL uses both light and heat to destroy the target structure/chromophores.

Laser, non-ablative, works by hearing up the underlying skin tissue with harming the surface. The heat generated by laser promotes collagen production and kills bacteria. The laser may be fractionated, that is it only treats a portion of any given section of skin it is exposed to. Typical non-ablative lasers include pulsed-dye lasers and fractional lasers.

Laser, ablative, directs short, concentrated pulsating beams of light that remove skin layer by layer. The two most commonly used ablative lasers are carbon dioxide (CO2) and erbium.

LED (light emitting diode) therapy uses varying wavelengths/spectrums/frequencies of light to help regenerate skin. LED therapy was originally developed to help wounds heal faster and to help regenerate damaged muscle tissue. Amber light stimulates collagen and elastin. Red light promotes circulation, reduces inflammation, and helps stimulate collagen. White light reduces inflammation. Blue light reduces oil production and kills bacteria. LED therapy is generally used to treat acne (blue light) and reduce the appearance of fine lines and wrinkles (red light).

Mircodermabrasian is a procedure that involves the use of superfine crystals, or diamond tipped wand on the skin to remove dead cells. Microdermabrasion can be used to improve the appearance of fine lines/wrinkles, minor hyperpigmentation, and enlarged pores.

Microneedling, also known as Collagen Induction Therapy, is a process that involves using an pen with tiny needles on the face to promote cell turnover. The result is improved skin texture, less noticeable pores, and long lasting collagen production.

Oxygenation of the skin involves the use of a pressurized stream of oxygen that delivers active ingredients to the skin. The ingredients can be, but are not limited to moisturizers, hyaluronic acid, antioxidants, peptides, vitamins, and the like.

Vein removal involves using a laser to decrease the appearance of spider veins on the skin.

The figures disclosed herein show before and after pictures of subjects treated using the disclosed treatment method combinations.

### Assessment of the Skin

With many of the above listed procedures, the results vary depending on the extent of the damages sought to be repaired. Furthermore, the patients skin color and type also affects the success of a treatment procedure. In general, the darker the skin, the more difficult it is to treat the skin condition.

Skin coloring can be classified using the Fitzpatrick scale. The Fitzpatrick scale includes six different skin types and colors with respect to the skin's toleration to the sun. Table 1, below, summarizes the Fitzpatrick scale.

TABLE 1
Fitzpatrick Scale

| Type | Features | Characteristics |
|---|---|---|
| I | pale white skin \| red or | always burns, never tans |
| | blond hair \| blue eyes \| freckles | |
| II | white or fair skin \| red or blond | usually burns, tans with |
| | hair \| blue, hazel, or green eyes | difficulty |
| III | cream white or fair skin \| any | gradually tans, sometimes |
| | eye or hair color | has a mild burn |
| IV | light brown skin | tans with ease, rarely burns |
| V | dark brown skin | tans very easily, very rarely |
| | | burns |
| VI | deeply pigmented dark brown | tans very easily, never burns |

The Fitzpatrick skin type is determined by a patients answer to the questions listed in Table 2, below. For each question, the answer will garner 0-4 points. The total number of point from each question is combined to determine the skin type. Skin type I has a total score of 0-7 points. Skin type II has a total score of 8-16 points. Skin type III has a total score of 17-25 points. Skin type IV has a total score of 25-30 points. Skin type V and VI have a total score of 30-40 points.

TABLE 2
Fitzpatrick scoring

| | Score | | | | |
|---|---|---|---|---|---|
| | 0 | 1 | 2 | 3 | 4 |
| What is the | Sandy red | Blond | Chestnut, | Dark | Black |
| natural color of | | | dark blond | brown | |
| your hair? | | | | | |
| What is the eye | Light blue, | Blue, | Blue | Dark | Brownish |
| color? | Gray, | Gray, | | Brown | Black |
| | Green | Green | | | |
| What is the | Reddish | Very pale | Pale with | Light | Dark |
| color of sun | | | beige tint | brown | brown |
| unexposed skin | | | | | |
| areas? | | | | | |
| How many | Many | Several | Few | Incidental | None |
| freckles on | | | | | |
| unexposed skin | | | | | |
| areas? | | | | | |
| What happens | Painful, | Blistering | Burns, | Rarely | Never |
| when you are in | redness, | followed | sometimes | burns | had a |
| the sun TOO | blistering, | by peeling | followed | | problem |
| long without | peeling | | by peeling | | |
| sunblock? | | | | | |
| How well do | Hardly or | Light | Reasonable | Tan very | Turn dark |
| you turn brown? | not at all | color tan | tan | easily | very |
| | | | | | quickly |
| Do you turn | Never | Seldom | Sometimes | Often | Always |
| brown within | | | | | |
| one day of sun | | | | | |
| exposure? | | | | | |
| How does your | Very | Sensitive | Normal | Very | Never |
| face respond to | sensitive | | | resistant | had a |
| the sun? | | | | | problem |
| When did you | More than | 2-3 month | 1-2 months | Less than | Less than |
| last expose | 3 months | ago | ago | 1 month | 2 weeks |
| yourself to the | ago | | | ago | ago |
| sun or artificial | | | | | |
| sun treatments? | | | | | |
| Do you expose | Never | Hardly | Sometimes | Often | Always |
| the area to be | | ever | | | |
| treated to the | | | | | |
| sun? | | | | | |

Total

The skin is evaluated to look for signs of damage and/or any of the conditions mentioned above. Sun damage often presents as mild redness and enlarged pores. The color of melasma, freckles, age spots are noted. Typically, a lighter brown color indicates deeper damage and can be difficult to treat. The darker brown color tends to be more superficial and thus more reactive to treatment.

The patient's lifestyle is also assessed to look for factors contributing to skin damage. For example, does the patient spend a lot of time outdoors? Does the patient smoke? Is the patient obese? Does the patient exercise? Does the patient have a high stress job or stressful home environment? Is the patient pregnant or plan to become pregnant in the near future? What medications and/or supplements is the patient taking? Lifestyle factors also provide an indication of how long treatment results might last.

A treatment plan is devised based on the patients Fitzpatrick skin type, skin conditions, and lifestyle. In one embodiment, the treatment plan includes at least two different skin treatments described above. In another embodiment, the treatment plan includes at least three different skin treatments described above. In yet another embodiment, the treatment plan includes at least four different skin treatments described above. In yet another embodiment, the treatment plan includes at least five different skin treatments described above.

In one embodiment, the treatment plan includes at least one treatment that targets the surface of the skin and at least one treatment that targets below the surface of the skin. In another embodiment, the treatment plan includes at least two treatments that targets the surface of the skin and at least one treatment that targets below the surface of the skin. In this embodiment, the second treatment that targets the surface of the skin may be done three days to six to seven weeks after the first treatment that targets the surface of the skin and the treatment that targets below the surface of the skin.

In one embodiment the treatment plan includes a non-ablative laser and a chemical peel. In another embodiment the treatment plan includes a non-ablative laser and a microdermabrasion. In another embodiment the treatment plan includes a non-ablative laser and microneedling. In another embodiment the treatment plan includes a non-ablative laser and oxygenation.

In one embodiment the treatment plan includes an LED therapy and a chemical peel. In another embodiment the treatment plan includes an LED therapy and a microdermabrasion. In another embodiment the treatment plan includes an LED therapy and microneedling. In another embodiment the treatment plan includes an LED therapy and oxygenation.

In one embodiment the treatment plan includes intense pulse light therapy and a chemical peel. In another embodiment the treatment plan includes intense pulse light therapy and a microdermabrasion. In another embodiment the treatment plan includes intense pulse light therapy and microneedling. In another embodiment the treatment plan includes intense pulse light therapy and oxygenation.

In another embodiment, the treatment plan includes an extraction and oxygen treatment. In another embodiment, the treatment plan includes an oxygen treatment and microneedling. In another embodiment the treatment plan includes microdermabrasion and microneedling.

In another embodiment, the treatment plan includes a blue light therapy and a facial. In another embodiment, the treatment plan includes a CO2 laser and a fractional. In another embodiment, the treatment plan includes a dermaplane and a peel. In another embodiment, the treatment plan includes a hydrafacial and a red light therapy. In another embodiment, the treatment plan includes a hydrafacial and a blue light therapy. In another embodiment, the treatment plan includes a laser and a peel. In another embodiment, the treatment plan includes Levulan IPL and blue light therapy. In another embodiment, the treatment plan includes a microneedle and a chemical peel. In another embodiment, the treatment plan includes a microneedle and PRP.

With any of the above embodied treatment plans, the plan can also include a dermaplane done about three days to 6-7 weeks after other skin treatment(s). For treatments involving microneedling, chemical peel, or ablative laser treatment, a dermaplane can reduce the time needed to achieve the “finished” look, especially in older subjects whose skin naturally takes longer to heal.

### EXAMPLES

The examples below use a Limelight laser made by Cutera, Inc. and Rejuvenize Peel made by SkinMedica, an Allergan company. The Limelight laser is an intense pulse light laser. Rejuvenize Peel is a self-neutralizing peel. Other lasers and chemical peels can be used with the settings adjusted to provide similar wavelengths/energy and peel depths.

### Example 1

In this example, the treatment plan is designed to reduce the effects of aging and photoaging. This treatment plan was for a Caucasian woman, Fitzpatrick type 1-2. The skin was numbed for 20 minutes with BPO (20% benzocaine/8% lidocaine/8% tetracaine (PL Asticized)). Ultrasound gel was applied to the skin. The skin was treated with the Limelight laser using the B-18 Sun Mode setting. The treated skin was wiped down with a lukewarm towel. Rejuvenize Peel was applied to the skin and the peel self-neutralized in about 6 hours. For the next 7 days, the subject used a gentle cleanser and moisturizer. On the 7^{th} day after the laser/peel treatment, the subject underwent a dermaplane treatment on the treated skin.

### Example 2

The same treatment plan as Example 1, but for a subject with a Fitzpatrick 1 skin type. For this skin type the Limelight laser is set to B-19 with sun mode on.

### Example 3

The same treatment plan as Example 1, but for a subject with a Fitzpatrick 2 skin type. For this skin type the Limelight laser is set to B-18 with sun mode on.

### Example 4

The same treatment plan as Example 1, but for a subject with a Fitzpatrick 3 skin type. For this skin type the Limelight laser is set to B-17 no sun mode.

### Example 5

The same treatment as plan as Example 1, but for a subject with a Fitzpatrick 4 skin type. For this skin type the Limelight laser is set to B-14-15 no sun mode. This skin type is difficult to treat and should be treated with caution. The Limelight laser should only treat the damaged areas.

### Example 6

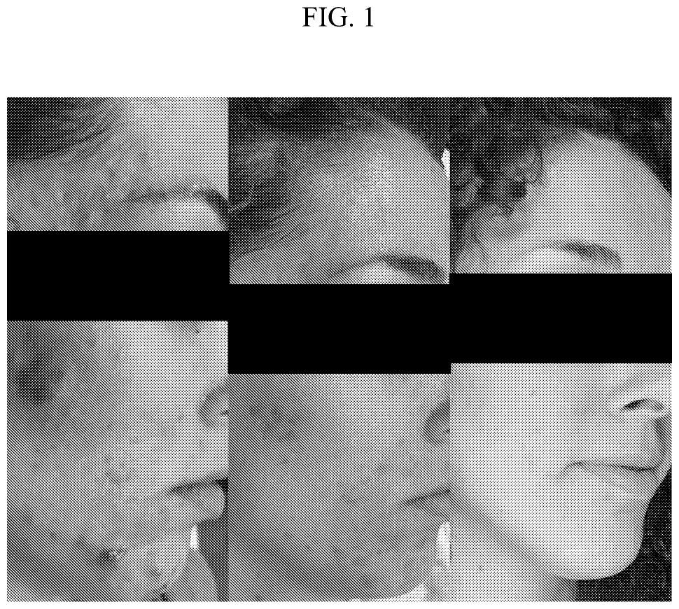

A subject with severe acne sought treatment. The subject underwent a treatment combination of microneedle and Rejuvenate peel and used Vivant® skin care products post treatment. Two months after treatment, the subject underwent a second treatment combination of microneedle and Rejuvenate peel. FIG. 1 shows the before and after images. The image on the right was taken about 6 months after the first treatment.

### Example 7

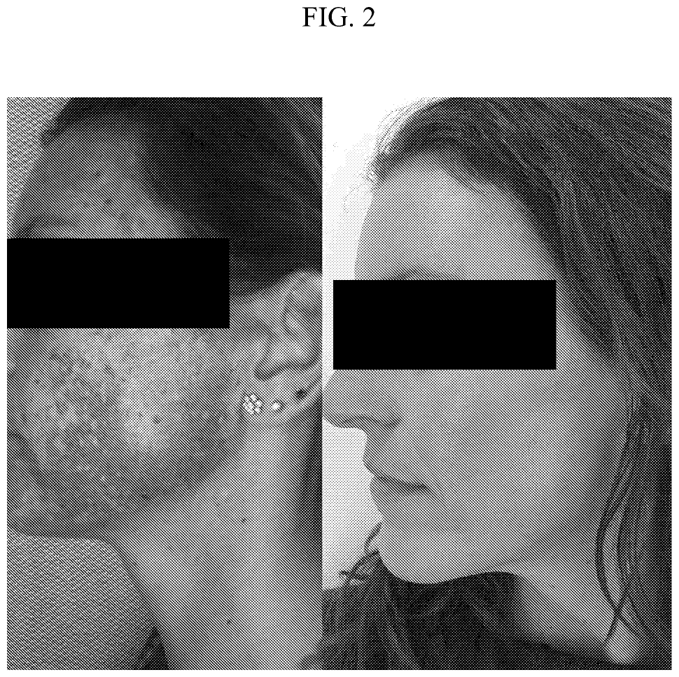

A subject with severe hyperpigmentation/melasma sought treatment. The subject underwent a treatment combination of Limelight laser and Rejuvenate peel. The subject underwent a dermaplane treatment several days after the laser/peel treatment. FIG. 2 shows before and after images of the subject. The picture on the right was taken 7 days after the laser/peel treatment.

### Example 8

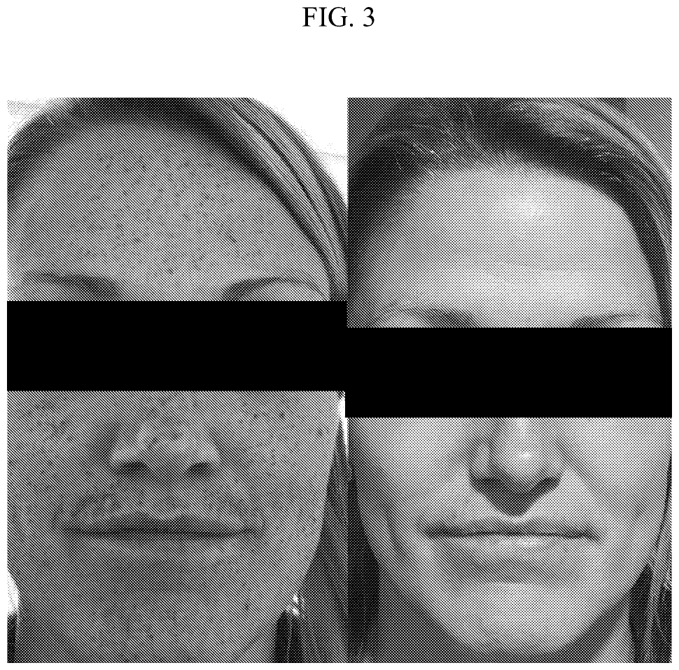

A subject with hyperpigmentation/freckles sought treatment. The subject underwent a treatment combination of Limelight laser and Rejuvenate peel. The subject underwent a dermaplane treatment several days after the laser/peel treatment. FIG. 3 shows before and after images of the subject. The picture on the right was taken 7 days after the laser/peel treatment.

### Example 9

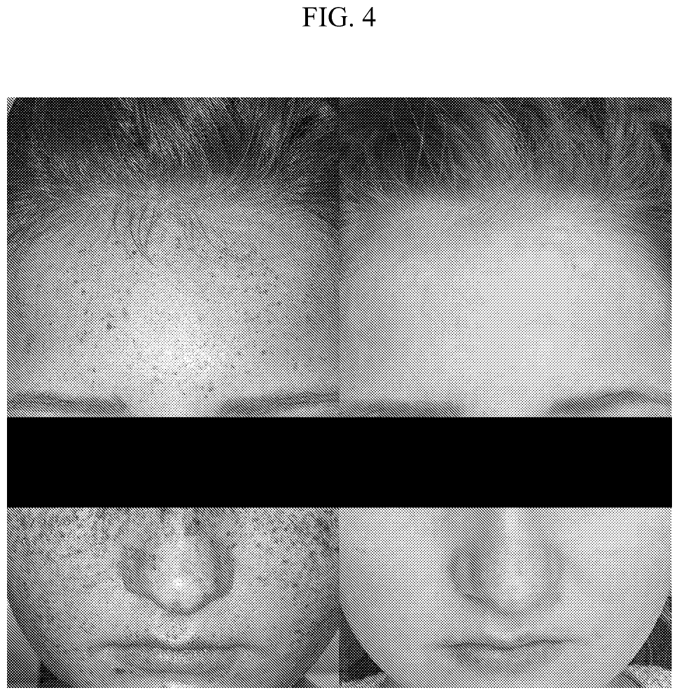

A subject with hyperpigmentation/freckles/melasma sought treatment. The subject underwent a treatment combination of Limelight laser and Rejuvenate peel. The subject underwent a dermaplane treatment several days after the laser/peel treatment. FIG. 4 shows before and after images of the subject. The picture on the right was taken 7 days after the laser/peel treatment.

### Example 10

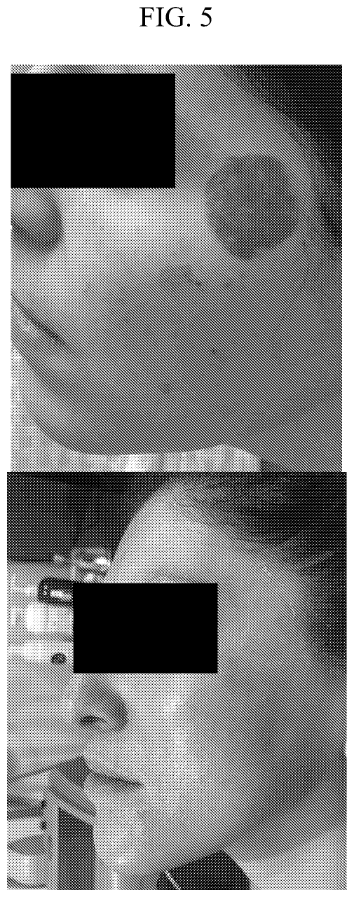

A subject with hyperpigmentation/melasma sought treatment. The subject underwent a treatment combination of Limelight laser and Rejuvenate peel. The subject underwent a dermaplane treatment several days after the laser/peel treatment. FIG. 5 shows before and after images of the subject. The picture on the bottom was taken 7 days after the laser/peel treatment.

The examples set forth above are provided to give those of ordinary skill in the art a complete disclosure and description of how to make and use embodiments of the compositions, and are not intended to limit the scope of what the inventors regard as their invention. Modifications of the above-described modes (for carrying out the invention that are obvious to persons of skill in the art) are intended to be within the scope of the following claims. All publications, patents and patent applications cited in this specification are incorporated herein by reference as if each such publication, patent or patent application were specifically and individually indicated to be incorporated herein by reference.

Many alterations and modifications may be made by those having ordinary skill in the art without departing from the spirit and scope of the embodiments disclosed and described herein. Therefore, it is understood that the illustrated and described embodiments have been set forth only for the purposes of examples and that they are not to be taken as limiting the embodiments as defined by the following claims. For example, notwithstanding the fact that the elements of a claim are set forth below in a certain combination, it must be expressly understood that the embodiments include other combinations of fewer, more or different elements, which are disclosed above even when not initially claimed in such combinations.

The definitions of the words or elements of the following claims are, therefore, defined in this specification to not only include the combination of elements which are literally set forth. It is also contemplated that an equivalent substitution of two or more elements may be made for any one of the elements in the claims below or that a single element may be substituted for two or more elements in a claim. Although elements may be described above as acting in certain combinations and even initially claimed as such, it is to be expressly understood that one or more elements from a claimed combination can in some cases be excised from the combination and that the claimed combination may be directed to a subcombination or variation of a subcombination(s).

Furthermore, to the extent that the term “having,” “includes,” or “wherein” is used in either the detailed description or the claims, such term is intended to be inclusive in a manner similar to the term “comprising” as “comprising” is interpreted when employed as a transitional word in a claim.

While various embodiments of the present invention have been described above, it should be understood that they have been presented by way of example only, and not of limitation. Likewise, the various diagrams may depict an example architectural or other configuration for the invention, which is done to aid in understanding the features and functionality that can be included in the invention. The invention is not restricted to the illustrated example architectures or configurations, but the desired features can be implemented using a variety of alternative architectures and configurations. Indeed, it will be apparent to one of skill in the art how alternative functional, logical or physical partitioning and configurations can be implemented to implement the desired features of the present invention. Also, a multitude of different constituent module names other than those depicted herein can be applied to the various partitions. Additionally, with regard to flow diagrams, operational descriptions and method claims, the order in which the steps are presented herein shall not mandate that various embodiments be implemented to perform the recited functionality in the same order unless the context dictates otherwise.

Although the invention is described above in terms of various exemplary embodiments and implementations, it should be understood that the various features, aspects and functionality described in one or more of the individual embodiments are not limited in their applicability to the particular embodiment with which they are described, but instead can be applied, alone or in various combinations, to one or more of the other embodiments of the invention, whether or not such embodiments are described and whether or not such features are presented as being a part of a described embodiment. Thus the breadth and scope of the present invention should not be limited by any of the above-described exemplary embodiments.

Terms and phrases used in this document, and variations thereof, unless otherwise expressly stated, should be construed as open ended as opposed to limiting. As examples of the foregoing: the term “including” should be read as meaning “including, without limitation” or the like; the term “example” is used to provide exemplary instances of the item in discussion, not an exhaustive or limiting list thereof; the terms “a” or “an” should be read as meaning “at least one,” “one or more” or the like; and adjectives such as “conventional,” “traditional,” “normal,” “standard,” “known” and terms of similar meaning should not be construed as limiting the item described to a given time period or to an item available as of a given time, but instead should be read to encompass conventional, traditional, normal, or standard technologies that may be available or known now or at any time in the future. Likewise, where this document refers to technologies that would be apparent or known to one of ordinary skill in the art, such technologies encompass those apparent or known to the skilled artisan now or at any time in the future.

A group of items linked with the conjunction “and” should not be read as requiring that each and every one of those items be present in the grouping, but rather should be read as “and/or” unless expressly stated otherwise. Similarly, a group of items linked with the conjunction “or” should not be read as requiring mutual exclusivity among that group, but rather should also be read as “and/or” unless expressly stated otherwise. Furthermore, although items, elements or components of the invention may be described or claimed in the singular, the plural is contemplated to be within the scope thereof unless limitation to the singular is explicitly stated.

The presence of broadening words and phrases such as “one or more,” “at least,” “but not limited to” or other like phrases in some instances shall not be read to mean that the narrower case is intended or required in instances where such broadening phrases may be absent. The use of the term “module” does not imply that the components or functionality described or claimed as part of the module are all configured in a common package. Indeed, any or all of the various components of a module, whether control logic or other components, can be combined in a single package or separately maintained and can further be distributed across multiple locations.

1. A method comprising the steps of:
(a) evaluating a skin of a subject for a skin condition;
(b) using a light to treat a portion of the skin;
(c) using a chemical peel to treat a portion of the skin; and
(d) exfoliating the treated areas of the skin;
wherein the exfoliating step occurs at least five days after steps (b) and (c).

2. A method comprising the steps of:
(a) evaluating a skin of a subject for a skin condition;
(b) using a laser to treat a portion of the skin;
(c) using a chemical peel to treat a portion of the skin; and
(d) exfoliating the treated areas of the skin;
wherein the exfoliating step occurs at least five days after steps (b) and (c).

3. The method of claim 1 or 2, wherein the skin condition is at least one of the following: acne, actinic keratosis, atopic dermatitis, broken capillaries, cherry anginomas, folliculitis barbae, pseudofolliculitis barbae, hyperpigmentation, keratosis pilaris, age spots, melisma, nevus, psoriasis, rosacea, scars, seborrheic dermatitis, skin tags, spider veins, or wrinkles.

4. The method of claim 1 or 2, wherein the skin condition is due to aging or photoaging.

5. The method of claim 1 or 2, wherein the evaluating of the skin further includes grading the skin on the following factors: age, ethnicity, pigmentation, texture, tone, and lifestyle.

6. The method of claim 1 or 2, wherein the evaluating of the skin further includes determining the Fitzpatrick type.

7. The method of claim 1 or 2, wherein the laser is a non-ablative laser.

8. The method of claim 1, wherein the light is intense pulse light.

9. The method of claim 8, wherein the intense pulse light operates in a wavelength range of 400 nanometers to 1200 nanometers.

10. The method of claim 1 or 2, wherein the chemical peel is a self-neutralizing peel.

11. The method of claim 1 or 2, wherein the exfoliating step comprises a dermaplane.

12. A method comprising the steps of:
(a) evaluating a skin of a subject for a skin condition resulting from aging or photoaging;
(b) determining a Fitzpatrick type of the skin of the subject;
(c) using an intense pulse light to treat a portion of the skin with the skin condition;
(d) using a self-neutralizing chemical peel to treat the portion of the skin with the skin condition; and
(e) using a dermaplane on the treated portion of the skin;
wherein the step (e) occurs at least five days after steps (c) and (d).

13. The method of claim 12, wherein the skin condition is melasma.

14. The method of claim 12, wherein intense pulse light wavelength has a mid-range 560 nanometer peak with a pulse duration time of 5-29 milliseconds and a fluence between 14-18 joules per centimeter squared.

15. A method for improving the appearance of skin comprising the steps of:
(a) evaluating a skin of a subject for a skin condition;
(b) using a treatment that targets below a surface of the skin to treat the portion of the skin;
(c) using a treatment that targets the surface of the skin to treat a portion of the skin and
(d) exfoliating the treated areas of the skin;
wherein the exfoliating step occurs at least five days after steps (b) and (c).

16. The method of claim 15, wherein the skin condition is at least one of the following: acne, actinic keratosis, atopic dermatitis, broken capillaries, cherry anginomas, folliculitis barbae, hyperpigmentation, keratosis pilaris, age spots, melisma, nevus, psoriasis, rosacea, scars, seborrheic dermatitis, skin tags, spider veins, or wrinkles.

17. The method of claim 15, wherein the treatment that targets the surface of the skin includes chemical peels, dermaplane, extractions, laser, microdermabrasion, microneedling, or oxygenation.

18. The method of claim 15, wherein the treatment that targets below the surface of the skin includes intense pulse light therapy, laser, or light emitting diode therapy.
